(19) United States
(12) Patent Application Publication
(10) Pub. No.: US 2019/0322974 A1
(43) Pub. Date: Oct. 24, 2019

(54) CULTURE CONTAINER WITH IN VIVO-LIKE ENVIRONMENT AND CULTURE DISH INCLUDING THE SAME

(71) Applicant: INUI MEDICAL LIMITED, Kooriyama-shi, Fukushima (JP)

(72) Inventors: Hiroaki INUI, Kooriyama (JP); Jinji MIZUNO, Kooriyama (JP); Eiko KIKUCHI, Kooriyama (JP); Kaori NOGUCHI, Kooriyama (JP); Yuri TANJI, Kooriyama (JP); Miki HAMABATA, Kooriyama (JP); Chiho KOTSUZUMI, Kooriyama (JP)

(21) Appl. No.: 16/302,998

(22) PCT Filed: Mar. 29, 2018
(86) PCT No.: PCT/JP2018/013349

§ 371 Date: Nov. 19, 2018

(30) Foreign Application Priority Data
Mar. 31, 2017 (JP) 2017-071372

(51) Int. Cl. C12M 1/12; C12M 1/02

(52) U.S. Cl. CPC C12M 25/06; C12M 27/00; C12M 25/04

(57) ABSTRACT

A culture container is provided that has an in vivo-like environment for culturing cells or embryos. The culture container includes rooms for individually receiving the cells or the embryos, wherein two or more rooms are connected via a space allowing passage of culture solution but preventing passage of cells or embryos; and a culture dish including the culture container. The culture dish is provided for supplying nutrient components and so forth as well as removing and discharging waste products. The culture dish includes a reserve tank and pathway for stably feeding fresh culture solution, and a waste tank.

### CROSS-REFERENCE

The present application claims priority based on Japanese Patent Application No. 2017-071372 filed in Japan on Mar. 31, 2017, the contents of which are incorporated herein in their entirety. Further, the entire contents of all patents, patent applications, and literatures cited in the present application are incorporated herein.

### BACKGROUND

### Technical Field

The present invention relates to a culture container for culturing a biomaterial such as cells or embryos, and a culture dish including the culture container.

### Related Art

Cells or embryos (collectively referred to as “cells, etc.” where appropriate) are conventionally cultured under a condition that does not deteriorate the property thereof. For example, in case of in vitro fertilization for mammals including humans, an egg is collected from a mother and fertilized, and the fertilized egg is cultured for a given period and then transplanted into the mother or another mother.

A known example of culture containers for cells, etc. is disclosed in Japanese Patent Laid-Open No. 2010-200748. The culture container disclosed in JP 2010-200748 includes a bottom wall and a side wall for culturing cells requiring individual control, wherein a cell receiving section including concaves is disposed in the bottom wall, four or more of the concaves are close to each other, the wall surface of each concave includes an inclined surface rising from the lowest position of the concave to the outer periphery side of the concave, and the spacing among the concaves close to each other is 1 mm or less. Use of such a culture container allows efficient outline extraction for cells in a photographic image of cultured cells, advantageously facilitating automatic discrimination of cultured cells.

It is important in culturing cells, etc. to keep the cells, etc. under conditions as close to those in a living body as possible. If a concave opening only in the upper side is used as a region for receiving cells, etc., it is easy to accurately discriminate cells, etc. from each other, but the environmental conditions are far from those in the in vivo environment. It is widely known that cells, etc. are affected by substances secreted from other cells, etc. in a living body, and this phenomenon affects the quality of cultured cells, etc. as well (paracrine effect). In particular, the paracrine effect should be considered in culturing fertilized eggs.

The present invention was made to meet these requirements, and an object thereof is to provide a culture container with an in vivo-like environment allowing culture under conditions closer to those in the in vivo environment, and a culture dish including the culture container.

### SUMMARY

The present inventors have diligently studied to produce a culture environment for cells, etc. closer to that in the in vivo environment, and eventually completed a culture container achieving an environment in which culture solution can circulate through a plurality of rooms individually receiving cells, etc. therein and allowing each of the cells, etc. received in the individual room to move like self-rotation. Specific solutions to the problem are as follows.

(1) One embodiment of the present invention is a culture container with an in vivo-like environment for culturing cells or embryos, including a plurality of rooms capable of individually receiving the cells or the embryos therein, wherein two or more of the rooms are connected together via a space with a size allowing passage of culture solution but not allowing passage of the cells or the embryos.

(2) In the container with an in vivo-like environment of another embodiment of the present invention, the rooms may be concaves open on one side, and the culture container may include slits each leading from an opening in a side wall of one of the concaves, the opening ranging from an upper position in the side wall to any position below, to an opening of a neighboring concave.

(3) In the culture container with an in vivo-like environment of another embodiment of the present invention, the slits may each have a length of 50% or less of a height of the concaves.

(4) In the culture container with an in vivo-like environment of another embodiment of the present invention, a volume of each of the rooms may be in a range of 10 to 50 nL.

(5) In another embodiment of the present invention, the culture container with an in vivo-like environment may further include: a reserve tank that feeds culture solution into the culture container; and a waste tank that stores culture solution discharged through the rooms.

(6) One embodiment of the present invention is a culture dish including any of the above culture containers with an in vivo-like environment, including: the culture container; a reserve tank that feeds culture solution into the culture container; and a waste tank that stores culture solution discharged from the culture container, wherein the reserve tank, the culture container, and the waste tank are disposed at different vertical positions such that the culture solution in an inner space of the reserve tank moves from the reserve tank to the waste tank through the culture container.

(7) The culture dish with an in vivo-like environment of another embodiment of the present invention may include a membrane between the reserve tank and the culture container, the membrane being capable of feeding the culture solution in the reserve tank to the culture container with a resistance to a gravity of the culture solution.

Cells to be cultured herein are preferably eukaryotic cells, more preferably animal cells such as mammalian cells and insect cells or plant cells, and even more preferably mammalian cells, though the cells are not limited thereto and may be any culturable cells. Cells or embryos can be suitably collected from, for example, humans; domestic animals such cattle, pigs, goats, and sheep; experimental animals (e.g., mice, rats, rabbits); and wild animals. Examples of the cells include undifferentiated cells including sperms, oocytes, amniotic mesenchymal cells, unfertilized eggs, fertilized eggs, embryonic cells, embryonic stem cells (ES cells), hematopoietic stem cells, mesenchymal stem cells, neural stem cells, cancer stem cells, and induced pluripotent stem cells (iPS cells); and differentiated cells including intimal cells such as endometrium cells, epithelial cells such as oviductal epithelial cells, amniotic epithelial cells, and biliary epithelial cells, fibroblasts, endothelial cells such as sinusoidal endothelial cells and vascular endothelial cells, and hepatocytes. Preferred are undifferentiated cells, and more preferred are undifferentiated germ cells such as sperms, oocytes, amniotic mesenchymal cells, unfertilized eggs, fertilized eggs, embryonic cells, and embryonic stem cells (ES cells). Embryos to be cultured herein are not limited and may be any culturable embryos, and preferred examples thereof include pronuclear stage embryos, early embryos, and blastocysts.

### Advantageous Effects of Invention

The present invention enables culturing cells or embryos under conditions closer to those in the in vivo environment.

### BRIEF DESCRIPTION OF DRAWINGS

FIG. 1 shows a plan view of a culture container of a first embodiment of the present invention.

FIG. 2A shows a cross-sectional view of the culture container of FIG. 1 along the line A-A and an enlarged cross-sectional view of a part C thereof, and FIG. 2B shows a cross-sectional view of the culture container along the line B-B.

FIG. 3A shows a cross-sectional view of the culture container of FIG. 2 including fertilized eggs received therein and an enlarged cross-sectional view of a part C thereof, and FIG. 3B shows an enlarged cross-sectional view of the part C while culture solution is flowing through a plurality of concaves.

FIG. 4 shows a cross-sectional view of a culture container of a second embodiment of the present invention as in FIG. 2A, and an enlarged cross-sectional view of a part C thereof.

FIGS. 5A and 5B show plan views of variations of the culture container of FIG. 4.

FIG. 6A shows a plan view of a culture container of a third embodiment of the present invention, and

FIG. 6B shows a vertical cross-sectional view along the line A-A of the plan view in culturing with the culture container.

FIG. 7 shows a simple exploded perspective view of a culture dish of a first embodiment of the present invention.

FIG. 8A shows a vertical cross-sectional view of the culture dish of FIG. 7, and FIG. 8B shows the same cross-sectional view during culture and an enlarged cross-sectional view of a part G thereof.

FIG. 9A shows a vertical cross-sectional view of a culture dish of a second embodiment of the present invention as in FIG. 8A, and FIG. 9B shows an enlarged cross-sectional view of a feed pipe.

FIGS. 10A-10C show results of Experimental Examples (1) to (3), where the graph in FIG. 10A shows development rates (%) with different culture containers, the graph in FIG. 10B shows cell counts for different cells with different culture containers, and the graph in FIG. 10C shows pregnancy rates (%) in recipient females and offspring productivities (%) with different culture containers.

FIGS. 11A-11C show results of Experimental Examples (4) to (6), where the graph in FIG. 11A shows development rates (%) with different culture containers, the graph in FIG. 11B shows cell counts for different cells with different culture containers, and the graph in FIG. 11C shows pregnancy rates (%) in recipient females and offspring productivities (%) with different culture containers.

### DETAILED DESCRIPTION

Embodiments of the culture container of the present invention with an in vivo-like environment and the culture dish including the culture container will be described with reference to the drawings. Embodiments described below are not intended to limit inventions of individual claims described in CLAIMS, and each of the components and combinations thereof described in the embodiments is not necessarily essential for the solutions by the present invention.

### 1. Culture Container

### First Embodiment

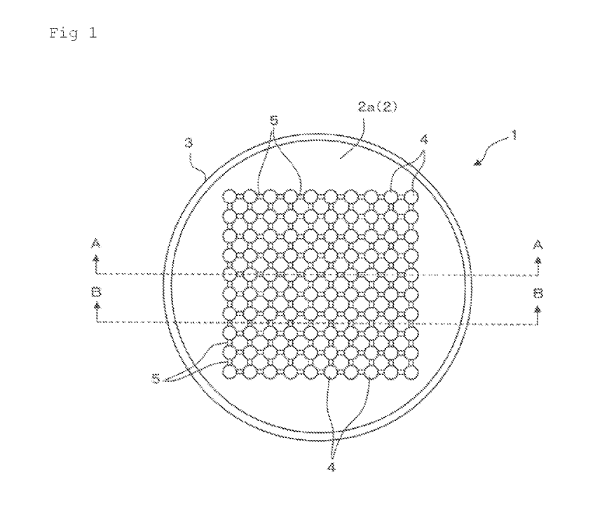

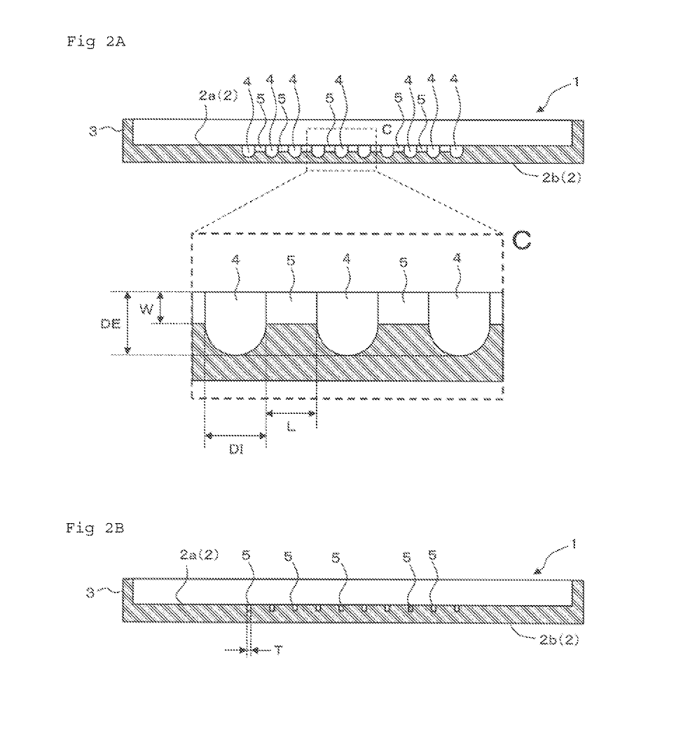

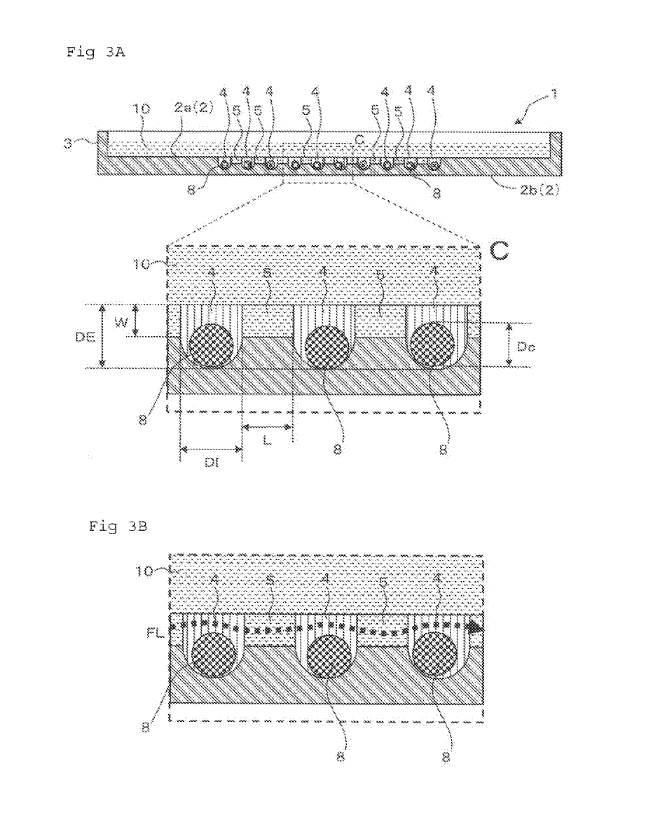

FIG. 1 shows a plan view of a culture container of a first embodiment of the present invention. FIG. 2A shows a cross-sectional view of the culture container of FIG. 1 along the line A-A and an enlarged cross-sectional view of a part C thereof, and FIG. 2B shows a cross-sectional view of the culture container along the line B-B. FIG. 3A shows a cross-sectional view of the culture container of FIG. 2 including fertilized eggs received therein and an enlarged cross-sectional view of a part C thereof, and FIG. 3B shows an enlarged cross-sectional view of the part C while culture solution is flowing through a plurality of concaves.

The culture container 1 with an in vivo-like environment (simply referred to as “culture container” hereinafter) of the first embodiment is a container for culturing fertilized eggs 8, an example of cells or embryos, and has a form like a Petri dish. More specifically, the culture container 1 includes a circular bottom plate 2 and a cylindrical side wall 3 protruding from the periphery of the bottom plate 2 toward the front side of the page. The shape of the bottom plate 2 is not limited to a circle, and may be a triangle, a rectangle, a polygon with five or more angles, an ellipse, or an irregular shape. In a surface 2a of the culture container 1 in the front side of the page, a plurality of concaves 4 sinking toward a back surface 2b of the bottom plate 2 are provided. The concaves 4 correspond to rooms capable of individually receiving fertilized eggs 8 therein. Each of the concaves 4 is a concave region like a cup open on the front side of the page (in one direction) of FIG. 1. The shape of the opening top of each concave 4, which is a circle in the present embodiment, may be a triangle, a rectangle, a polygon with five or more angles, an ellipse, or an irregular shape, similarly to the shape of the bottom plate 2.

In the present embodiment, the culture container 1 includes concaves 4 as 10 rows×10 columns, or 100 concaves 4 in total. However, the numbers of rows and columns and the total number of the concaves 4 are not limited to the exemplified ones. In culturing human fertilized eggs 8, 2 to 20 concaves 4 in the culture container 1 may be formed. In culturing fertilized eggs 8 derived from a domestic animal such as cattle, a pig, sheep, and a goat, or an experimental animal such as a mouse, a rat, and a rabbit, 30 to 100 concaves 4 in the culture container 1 may be formed. The disposition pattern of the concaves 4 is not limited to the grid pattern.

In FIG. 1, concaves 4 neighboring each other in both latitudinal and longitudinal directions are connected together via slits 5. More specifically, the side surface of one concave 4 and the side surface of another concave 4 are communicating via a slit 5. The slit 5 is an example of a space allowing passage of culture solution 10 but not allowing passage of fertilized eggs 8. In the present embodiment, the concaves 4 at the corners among the plurality of concaves 4 disposed in a grid pattern are each connected to two slits 5. The concaves 4 in the sides except the corners are each connected to three slits 5. The other concaves 4 are each connected to four slits 5. In this manner, each concave 4 in the present embodiment is connected to other concaves 4 neighboring in the latitudinal direction and the longitudinal direction via two to four slits 5. It is preferred to cover the top of the culture solution 10 in the culture container 1 with oil as an upper layer to prevent drying.

As illustrated in FIG. 2A, each concave 4 preferably has a form including a curved bottom and a generally-vertical side wall extending therefrom. This is because the form allows a fertilized egg 8 in each concave 4 to freely rotate or rock to result in lower possibility of damaging and difficulty in escape from the concave 4. Each slit 5 is a space with a generally-cuboidal shape in the present embodiment. However, the shape of each slit 5 is not limited to a generally-cuboidal shape, and may be, for example, a wavily-deformed cuboid or a pyramid (the shape of a wedge).

Examples of the material constituting the culture container 1 include materials suitably formed from: polyamide; polyimide; cyclic olefin copolymer; polyolefin such as polyethylene, polypropylene, ethylene-propylene copolymer, and ethylene-vinyl acetate copolymer; polyester such as polyethylene terephthalate, polybutylene terephthalate, polyethylene naphthalate, and polybutylene naphthalate; synthetic resin including, as representative examples, polystyrene such as polystyrene and methacrylate-styrene copolymer; thermosetting elastomer such as silicone rubber, urethane rubber, isoprene rubber, ethylene-propylene rubber, ethylene-propylene-diene rubber, nitrile rubber, and styrene-butadiene rubber; or thermoplastic elastomer such as urethane elastomer, ester elastomer, styrenic elastomer, olefinic elastomer, butadiene elastomer, and fluoroelastomer, though the material constituting the culture container 1 is not limited thereto and may be any material which does not interfere with culture of cells or embryos. Except for any of the synthetic resins and rubbers, the culture container 1 may be constituted with, for example, glass; metal such as aluminum, aluminum alloy, and stainless steel; or ceramic such as aluminum oxide and silicon nitride. More preferred examples of the material of the culture container 1 include materials suitably formed from: polyamide; polyimide; cyclic olefin copolymer; polyolefin such as polyethylene, polypropylene, ethylene-propylene copolymer, and ethylene-vinyl acetate copolymer; polyester such as polyethylene terephthalate, polybutylene terephthalate, polyethylene naphthalate, and polybutylene naphthalate; synthetic resin, including, as representative examples, polystyrene such as polystyrene and methacrylate-styrene copolymer; thermosetting elastomer such as silicone rubber, urethane rubber, isoprene rubber, ethylene-propylene rubber, ethylene-propylene-diene rubber, nitrile rubber, and styrene-butadiene rubber; and thermoplastic elastomer such as urethane elastomer, ester elastomer, styrenic elastomer, olefinic elastomer, butadiene elastomer, and fluoroelastomer, though the material of the culture container 1 is not limited thereto and may be any material which does not interfere with culture of cells or embryos (fertilized eggs 8, as an example).

The diameter of the bottom plate 2 of the culture container 1 is preferably 4 to 15 mm, and more preferably 6 to 12 mm, though the diameter is not limited thereto. The diameter, DI of each concave 4, can be arbitrarily set according to the size of cells or embryos to receive therein, and is preferably 200 to 400 and more preferably 250 to 300 μm when human fertilized eggs 8 are received in the concaves 4. Similarly, the height (depth), DE of each concave 4, can be arbitrarily set according to the size of cells or embryos to receive therein, and is preferably 150 to 400 and more preferably 200 to 300 μm when human fertilized eggs 8 are received in the concaves 4. The inner volume of each concave 4 (excluding slits 5) can be arbitrarily set as well according to the size of cells or embryos to receive therein, and is preferably 10 to 50 nL, more preferably 15 to 30 nL, and the most preferably 20 nL when human fertilized eggs 8 are received in the concaves 4.

The length, L of each slit 5 (corresponding to the thickness of a thick part between neighboring concaves 4, 4), is preferably 5 to 200 The interval between fertilized eggs 8 is preferably 80 to 160 The width, T (thickness) of each slit 5, is preferably 2 to 40 and more preferably 5 to 30 though the width is not limited thereto and may be any width not allowing passage of human fertilized eggs 8 and allowing passage of the culture solution 10. Each slit 5 has a shape of a groove formed by cutting in the depth direction of the concaves 4, more specifically, has the shape of a groove formed by cutting from the opening top of a concave 4 to any depth above the bottom of the concave 4. In other words, each slit 5 is a slit leading from an opening in the side wall of a concave 4, the opening ranging from an upper position in the side wall to any position below, to a like opening in a neighboring concave 4. The depth, W of each slit 5, satisfies 0<W≤DE, and preferably satisfies 0<W≤DE/2, where DE denotes the depth of each concave 4. In this case, the slit 5 has a length of 50% or less of the depth, DE of each concave 4. The depth, W of each slit 5, more preferably satisfies the relation DE/3≤W≤DE/2. If the depth, W of each slit 5, is set in the range of 0 to ½ (0 to 50%), or even in the range of ⅓ to ½ (33 to 50%) of the depth, DE of each concave 4, a flow, FL of the culture solution 10, is generated in the upper part of each concave 4 as schematically illustrated in FIG. 3 (3B), and thus an environment can be constructed such that a fertilized egg 8 in each concave 4 only slightly rocks without excessive rolling.

A human fertilized egg 8 has a diameter (Dc) of approximately 100 to 200 If the depth, DE of each concave 4, is set to 200 to 300 μm, a space of 0 to 200 μm is present in the opening top side of each concave 4 including a human fertilized egg 8 received therein (however, the culture solution 10 is present in the space). If the depth, W of each slit 5, is set to 33 to 50% of the depth, DE of each concave 4, then W is approximately 6 to 150 Assuming that a human fertilized egg 8 often stays on the bottom of each concave 4, each slit 5 is present at a level of the upper half (northern hemisphere) of the fertilized egg 8 or a shallower position in that setting. Hence, the fertilized egg 8 only rotates or rocks in the concave 4, and is less likely to escape from the opening of the concave 4. Through formation of a space typified by such a slit 5, an environment can be constructed such that the culture solution 10 flows through neighboring concaves 4. Thereby, the paracrine effect during culture of fertilized eggs 8 can be enhanced. In addition, troubles including mix-up of fertilized eggs 8 are avoided since fertilized eggs 8 are individually received in the concaves 4 during culture.

Suitable for use as the culture solution 10 is a culture solution prepared by dissolving an inorganic salt, an energy source (e.g., sugar, amino acid, pyruvic acid, glycine, octanoic acid), a cytoprotective substance (e.g., polyvinyl alcohol, glycosaminoglycan including hyaluronan as a representative example), an antibiotic, and a bioactive substance (e.g., growth factors, cytokines) in ultrapure or re-distilled water. Examples of the culture solution 10 include culture solutions containing, as base culture solution, (4-(2-hydroxyethyl)-1-piperazineethanesulfonic acid (HEPES)-containing medium) containing: sodium chloride; potassium dihydrogen phosphate; potassium chloride; calcium chloride; magnesium sulfate heptahydrate; 19 amino acids; sodium hydrogen carbonate; disodium ethylenediaminetetraacetate dihydrate; gentamicin sulfate; polyvinyl alcohol; alanyl-L-glutamine; and D-glucose, a lactate such as sodium DL-lactate, and a pyruvate such as sodium pyruvate as energy sources. The culture solution 10 may contain one or more selected from sucrose, glucose, trehalose, dextran, Percoll, polyethylene glycol, polyvinyl alcohol, hyaluronan, fibronectin, polyvinylpyrrolidone, human serum albumin, and so forth. Very preferred culture solutions are, for example, KSOM (Lawitts J A, Biggers J D. Culture of preimplantation embryos. Methods Enzymol. 1993; 225: 153-64.) and G-TL (Vitrolife). The culture solution 10 can be serum-free.

### Second Embodiment

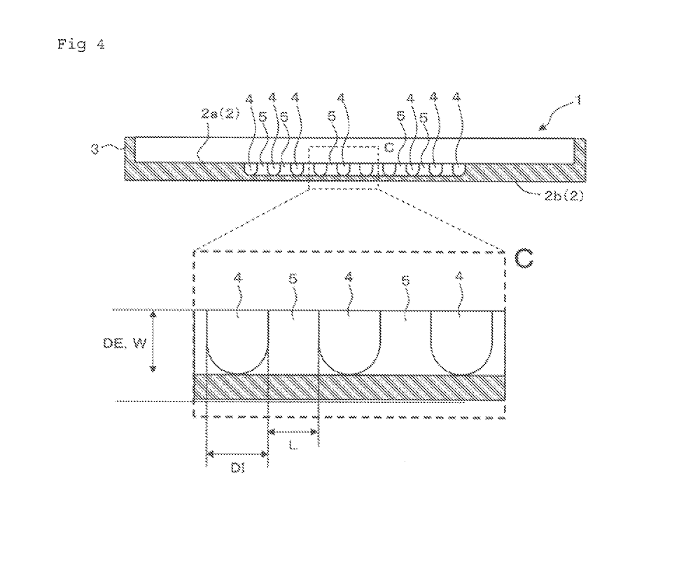

FIG. 4 shows a cross-sectional view of a culture container of a second embodiment of the present invention as in FIG. 2A, and an enlarged cross-sectional view of a part C thereof.

The culture container 1 of the second embodiment has the same structure as that of the culture container 1 of the first embodiment except that the form of each slit 5 differs from that of the culture container 1 of the first embodiment. Thus, in the following description, the description of the first embodiment is referred for configurations other than slits 5, and redundant description is omitted.

The culture container 1 of the second embodiment includes slits 5 having the same depth, W, as the depth, DE of concaves 4. The length, L, and width, T, of the slits 5 are in common with those in the first embodiment. Even though the depth, W of the slits 5, is the same as the depth, DE of the concaves 4, a fertilized egg 8 cannot move to a neighboring concave 4. This is because the width, T of each slit 5, is set smaller than the diameter of a fertilized egg 8, Dc. If the depth, W of the slits 5, is set larger than that in the first embodiment, the culture solution 10 flows through a deeper part of a concave 4 to a neighboring concave 4. It follows that the circulation of the culture solution 10 is enhanced. Considering comprehensively on various factors including the possibilities of damaging a fertilized egg 8 and slipping-out of a fertilized egg 8 from a concave 4, however, the depth of the slits 5 in the first embodiment is deemed to be more preferable in some interpretations.

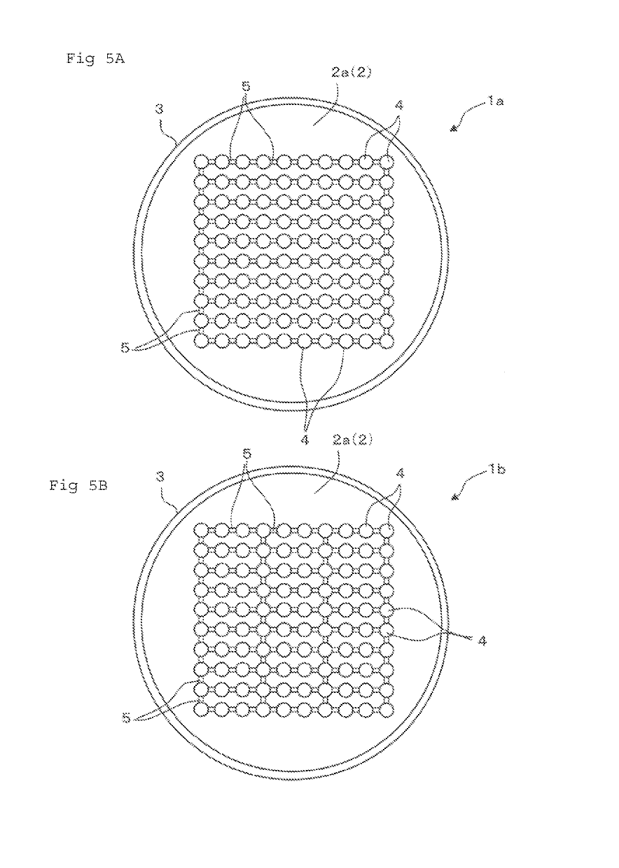

FIGS. 5A and 5B show plan views of variations of the culture container of FIG. 4.

As illustrated in FIG. 5A, the culture container 1a of the first variation is in common with the culture container 1 of FIG. 4 (or FIG. 1) in that the culture container 1a includes concaves 4 as 10 rows×10 columns, or 100 concaves 4 in total. However, the culture container 1a differs from the culture container 1 of FIG. 4 (or FIG. 1) in that slits 5 connecting concaves 4 only in the longitudinal direction are provided except in the periphery of a grid pattern of concaves 4.

Further, as illustrated in FIG. 5B, the culture container 1b of the second variation is in common with the culture container 1 of FIG. 4 (or FIG. 1) in that the culture container 1b includes the concaves 4 as 10 rows×10 columns, or 100 concaves 4 in total. However, the culture container 1b differs from the culture container 1 of FIG. 4 (or FIG. 1) in that slits 5 connecting concaves 4 in the latitudinal direction are provided in a grid pattern of concaves 4 at intervals of two columns.

The first variation and the second variation can be applied to the culture container 1 of the first embodiment. The slits 5 connecting concaves 4 can be not only in the complete grid connection pattern as in the first embodiment, but also in a modified pattern as illustrated in FIGS. 5A and 5B, and other modified patterns are acceptable. It is only required for each slit 5 to connect at least two concaves 4.

Each slit 5 is preferably a groove open toward the opening side of the concaves 4, as illustrated in the above embodiment. However, each slit 5 may be a through-hole connecting concaves 4 as a space with the opening side of the concaves 4 closed.

### Third Embodiment

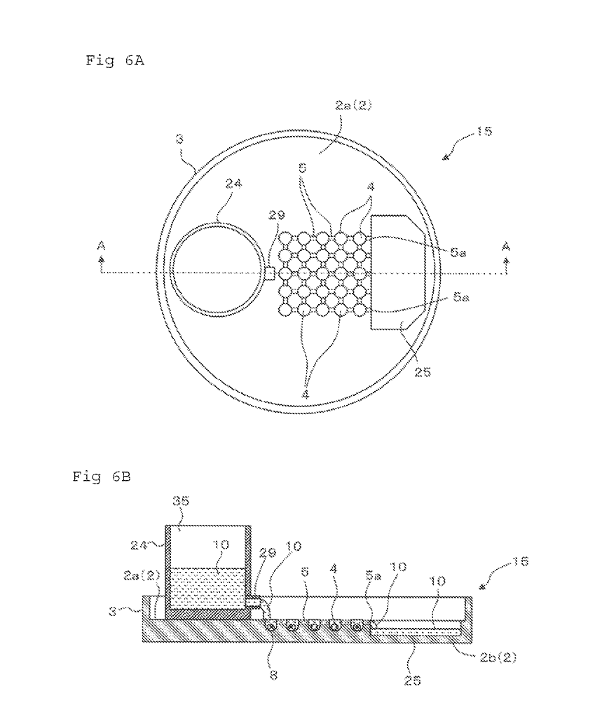

FIG. 6A shows a plan view of a culture container of a third embodiment of the present invention, and FIG. 6B shows a vertical cross-sectional view along the line A-A of the plan view in culturing with the culture container.

The culture container 15 of the third embodiment further includes, on a surface 2a of the culture container 15, a reserve tank 24 that is provided at a position out of the region where concaves 4 are disposed and feeds the culture solution 10 into the culture container 15, and a waste tank 25 that stores the culture solution 10 discharged through concaves 4. The culture container 15 includes concaves 4 as 5 rows×5 columns, or 25 concaves 4 in total. Slits 5 are formed as a grid pattern of 5 rows×5 columns to connect the concaves 4.

The waste tank 25 in the present embodiment is formed in a surface 2a in the front side of the bottom plate 2 as a concave region open on the surface 2a side. The culture container 15 and the reserve tank 24 are disposed on a region where the waste tank 25 is not disposed in the surface 2a. The reserve tank 24 is a cylindrical member capable of storing the culture solution 10 in an inner space 35 thereof. The reserve tank 24 is capable of feeding the culture solution 10 put in the inner space 35 onto the surface 2a through a feed pipe 29. The feed pipe 29 may include a valve to prevent the culture solution 10 from unintentionally flowing out onto the surface 2a.

The slits 5 communicate the concaves 4, and preferably lead to the waste tank 25 as well. The feed pipe 29, slits 5, and waste tank 25 are formed so that they are descending in the order presented. As a result, the culture solution 10 in the reserve tank 24 flows out of the feed pipe 29 onto the surface 2a, then moves into a concave 4 including a fertilized egg 8 received therein and further into another concave 4 through a slit 5, and finally arrives at an outlet 5a for the waste tank 25 and moves into the waste tank 25. In this way, the culture solution 10 can be sent from the reserve tank 24 through the culture container 15 into the waste tank 25 with use of gravity, even without any feeding apparatus such as a pump. The feeding rate of the culture solution 10 from the reserve tank 24 to the culture container 1 can be in the range of 1 to 20 nL/min, and more preferably in the range of 5 to 15 nL/min, though the feeding rate is not limited thereto.

The material constituting the reserve tank 24 is, for example, suitably formed from: polyamide; polyimide; cyclic olefin copolymer; polyolefin such as polyethylene, polypropylene, ethylene-propylene copolymer, and ethylene-vinyl acetate copolymer; polyester such as polyethylene terephthalate, polybutylene terephthalate, polyethylene naphthalate, and polybutylene naphthalate; synthetic resin including, as representative examples, polystyrene such as polystyrene and methacrylate-styrene copolymer; thermosetting elastomer such as silicone rubber, urethane rubber, isoprene rubber, ethylene-propylene rubber, ethylene-propylene-diene rubber, nitrile rubber, and styrene-butadiene rubber; or thermoplastic elastomer such as urethane elastomer, ester elastomer, styrenic elastomer, olefinic elastomer, butadiene elastomer, and fluoroelastomer, though the material constituting the reserve tank 24 is not limited thereto and may be any material which does not interfere with culture of cells or embryos (fertilized eggs 8, in an example). Alternatively to any of the synthetic resins and rubbers, the reserve tank 24 may be constituted with, for example, glass; metal such as aluminum, aluminum alloy, and stainless steel; or ceramic such as aluminum oxide and silicon nitride. More preferred examples of the material of the reserve tank 24 include polystyrene, silicone rubber, and glass.

The diameter of the bottom plate 2 of the culture container 15 is preferably 30 to 100 mm, and more preferably 35 to 71 mm, though the diameter is not limited thereto and may be any diameter allowing disposition of the reserve tank 24 and the waste tank 25. The height of the side wall 3 is preferably 7 to 15 mm, and more preferably 10 to 12 mm, though the height is not limited thereto as well.

### 2. Culture Dish

### First Embodiment

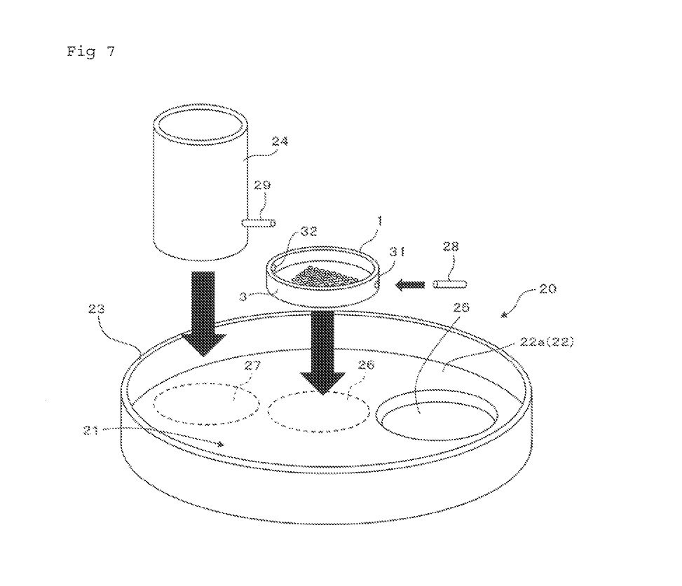

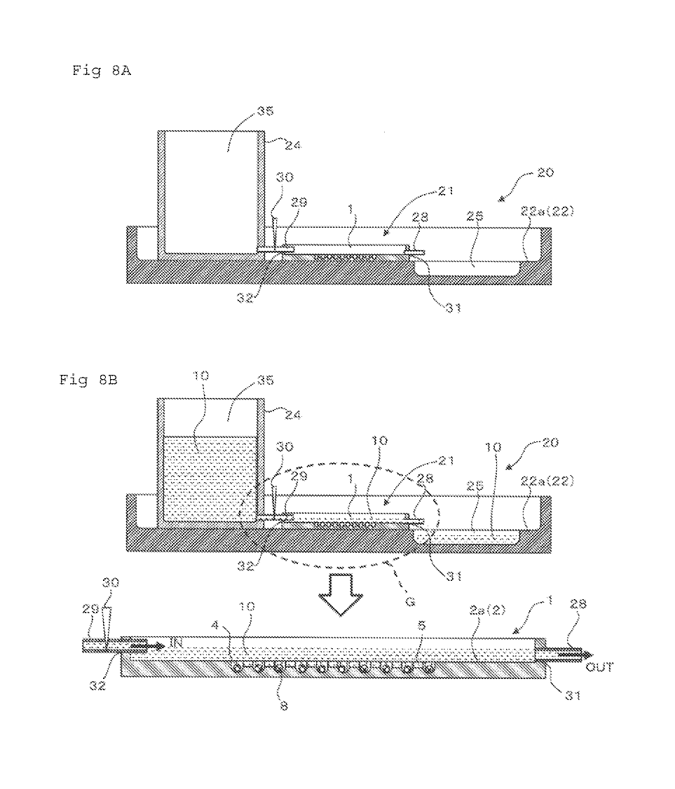

FIG. 7 shows a simple exploded perspective view of a culture dish of a first embodiment of the present invention. FIG. 8A shows a vertical cross-sectional view of the culture dish of FIG. 7, and FIG. 8B shows the same cross-sectional view during culture and an enlarged cross-sectional view of a part G thereof.

The culture dish 20 with an in vivo-like environment (simply referred to as “culture dish” hereinafter) includes: a culture container 1; a reserve tank 24 that feeds culture solution 10 into the culture container 1; and a waste tank 25 that stores the culture solution 10 discharged from the culture container 1. The reserve tank 24, the culture container 1, and the waste tank 25 in the culture dish 20 are disposed at different vertical positions such that the culture solution 10 in an inner space of the reserve tank 24 moves from the reserve tank 24 to the waste tank 25 through the culture container 1. Now, the detailed configuration of the culture dish 20 will be described.

The culture dish 20 includes a circular bottom plate 22 and a cylindrical side wall 23 protruding from the periphery of the bottom plate 22 toward the upper direction. The shape of the bottom plate 22 is not limited to a circle, and may be a triangle, a rectangle, a polygon with five or more angles, an ellipse, or an irregular shape. The culture container 1 and the reserve tank 24 can be disposed in a region 21 surrounded by the side wall 23 in the culture dish 20. The waste tank 25 in the present embodiment is formed in a surface 22a in the front side of the bottom plate 22 as a concave region open on the surface 22a side. The culture container 1 and the reserve tank 24 can be disposed in a region 26 and region 27, respectively, where the waste tank 25 is not disposed, in the surface 22a.

The culture container 1 includes through-holes 31 and 32 in the side wall 3. The through-hole 31 is a hole with a size allowing insertion of a discharge pipe 28 that discharges the culture solution 10 in the culture container 1 to the waste tank 25. The through-hole 31 is formed in the side wall 3 preferably at a vertical position as close to the surface 2a of the culture container 1 as possible. The through-hole 32 is a hole with a size allowing insertion of a feed pipe 29 that feeds the culture solution 10 in the reserve tank 24 into the culture container 1. The through-hole 32 is formed in the side wall 3 at a vertical position as close to the opening top of the culture container 1 (above the surface 2a) as possible. It follows that the vertical position of the feed pipe 29 inserted into the through-hole 32 is higher than that of the discharge pipe 28 inserted into the through-hole 31. As a result, the culture solution 10 can be sent from the reserve tank 24 through the culture container 1, etc. to the waste tank 25 with use of gravity, even without any feeding apparatus such as a pump. The feed pipe 29 is connected at a vertical position higher than that of the inner bottom surface of the reserve tank 24, and preferably disposed near the inner bottom surface. The discharge pipe 28 may be a member fixed in advance to either the culture container 1 or the reserve tank 24, or an independent member attachable to and detachable from both the culture container 1 and the reserve tank 24. The feed pipe 29 may be a member fixed in advance to the culture container 1, or an independent member attachable to and detachable from the culture container 1. The culture container 1 does not necessarily need to include the through-hole 32. If the feed pipe 29 is disposed above the side wall 3 of the culture container 1, formation of the through-hole 32 in the side wall 3 is not needed.

The reserve tank 24 is a cylindrical member capable of storing the culture solution 10 in the inner space 35 thereof. The feed pipe 29 preferably includes a valve 30 to prevent the culture solution 10 put in the inner space 35 from unintentionally flowing out of the feeding pipe 29. Although the discharge pipe 28 does not include a valve in the present embodiment, the discharge pipe 28 may include a valve.

As illustrated in FIG. 8B, when the culture container 1 including fertilized eggs 8 and the culture solution 10 received therein and the reserve tank 24 are disposed in the culture dish 20, and the culture solution 10 is put in the inner space 35 of the reserve tank 24 and the valve 30 is opened, the culture solution 10 in the reserve tank 24 enters the culture container 1 through the feed pipe 29, and replaces the culture solution 10 in the culture container 1. The culture solution 10 replaced is sent to the waste tank 25 through the discharge pipe 28, and stored therein. Since a plurality of concaves 4 in the culture container 1 is connected together via slits 5, the culture solution 10 can be move through a plurality of concaves 4. Substances discharged from a fertilized egg 8 in one concave 4 are sent to a fertilized egg 8 in each neighboring concave 4, and further sent to fertilized eggs 8 in the subsequent other concaves 4, together with the culture solution 10. The slits 5 contribute to enhancement of the paracrine effect. Although the concaves 4 are communicated with each other via the openings even without the slits 5, the degree of movement of the culture solution 10 and the discharged substances among the concaves 4 is smaller than that in the case with the slits 5. The feeding rate of the culture solution 10 from the reserve tank 24 to the culture container 1 can be in the range of 1 to 20 nL/min, and more preferably in the range of 5 to 15 nL/min, though the feeding rate is not limited thereto.

The material constituting the culture dish 20 can be selected from the options for the material of the culture container 1, though the material constituting the culture dish 20 is not limited thereto and may be any material which does not interfere with culture of cells or embryos.

The diameter of the bottom plate 22 of the culture dish 20 is preferably 30 to 100 mm, and more preferably 35 to 71 mm, though the diameter is not limited thereto and may be any diameter allowing disposition of the reserve tank 24 and the waste tank 25. The height of the side wall 23 is preferably 7 to 15 mm, and more preferably 10 to 12 mm, though the height is not limited thereto as well.

Although the culture container 1 is an object independent of the culture dish 20 in the present embodiment, the culture container 1 may be fixed to a generally-central portion of the culture dish 20. In this case, the culture dish 20 is regarded as a member including the culture container 1 with additional concaves outside thereof along the radial direction, and can be referred to as a culture container.

### Second Embodiment

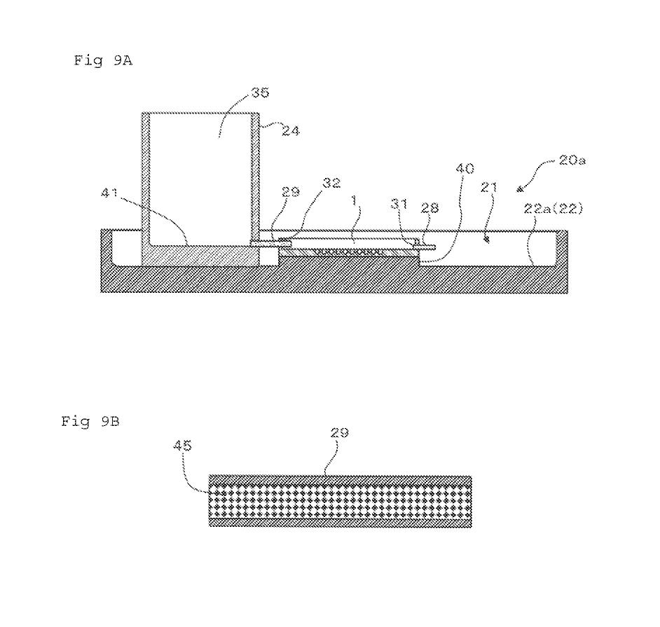

FIG. 9A shows a vertical cross-sectional view of a culture dish of a second embodiment of the present invention as in FIG. 8A, and FIG. 9B shows an enlarged cross-sectional view of a feed pipe.

The culture dish 20a of the second embodiment has the same structure as the culture dish 20 of the first embodiment except that a region 21 in the front of the container 1 in the culture dish 20 is utilized as a waste tank 25, and that a flow rate control unit is provided inside the feed pipe 29 instead of the valve 30. Thus, in the following description, the description of the first embodiment is referred for configurations in common with those in the first embodiment, and redundant description is omitted.

The culture dish 20a of the second embodiment includes a mount 40 protruding toward the upper direction in the generally-central portion. The culture container 1 is disposed on the mount 40. The reserve tank 24 has a bottom higher than that of the same tank 24 in the first embodiment. The vertical position of the feed pipe 29 is higher than that of the surface 2a of the culture container 1. The discharge pipe 28 is, on the other hand, positioned sufficiently higher than a surface 22a of the culture dish 20a. As a result, the culture solution 10 in the inner space 35 of the reserve tank 24 moves into the culture container 1 through the feed pipe 29, and then into the culture dish 20a through the discharge pipe 28, and stored therein. The mount 40 is not essential in the configuration. The mount 40 is unnecessary if the bottom plate 2 of the culture container 1 is sufficiently thick.

The feed pipe 29 includes, in the inside, a membrane 45 allowing control of the discharge of the culture solution 10 for gradual discharge. The membrane 45, which is disposed between the reserve tank 24 and the culture container 1, is a member capable of feeding the culture solution 10 in the reserve tank 24 to the culture container 1 with a resistance to a gravity thereof. Although the membrane 45 is filling a space over the length of the feed pipe 29 in the present embodiment, the membrane 45 may be present in a region over a length smaller than that of the feed pipe 29. The membrane 45 may be disposed at an end surface either in the reserve tank 24 side or in the culture container 1 side of the feed pipe 29.

The membrane 45 is a porous membrane consisting of a material of, for example, cellulose, polyester, cellulose-mixed ester, polyvinylidene fluoride, or polytetrafluoroethylene, and has a function to gradually pass the culture solution 10 through many pores. The thickness and pore size of the membrane 45 can be changed depending on conditions including the viscosity and feeding rate of the culture solution 10. The membrane 45 is a membrane for flow rate control, and capable of controlling the feeding rate of the culture solution 10 from the reserve tank 24 to the culture container 1, etc. preferably in the range of 0.1 to 200 nL/min, more preferably in the range of 5 to 15 nL/min. The position for installation and structure of a dam for flow rate control may be such that the dam is disposed in a flow path connecting the reserve tank and the culture container to narrow the middle of the flow path to give a restricted flow rate. The position for installation and specification of the membrane 45 for flow rate control can be such that the membrane 45 is disposed in a flow path connecting the reserve tank 24 and the culture container 1, where porous membrane filters (e.g., cellulose-mixed ester, polyvinylidene fluoride, polytetrafluoroethylene) for filtration sterilization with different pore diameters of 0.025 μm to 10 μm are appropriately used as the membrane 45 according to the composition, characteristics, viscosity, and so forth of the culture solution 10.

The membrane 45 can be provided not only in the feed pipe 29 for the reserve tank 24 included in the culture dish 20 or 20a, but also in the feed pipe 29 for the reserve tank 24 disposed in the above-described culture container 15.

### 3. Method for Producing Culture Container and Culture Dish

Each of the culture containers 1, 1a, 1b, and 15 and the culture dishes 20 and 20a (referred to as “culture container 1, etc.”, appropriately) can be produced as a form without connecting and add-on members including the reserve tank 24, the feed pipe 29, and the discharge pipe 28, through molding, combination of photolithography and transfer, or 3D-printing. In molding, it is preferred to feed an uncured curable resin composition or curable rubber composition into a mold and cure the composition in the mold, for example, through heating (involving pressurizing, in some cases) or UV irradiation. In this case, concaves 4 and slits 5 may be formed during molding, or formed through laser processing or machining after molding.

In photolithography and transfer, for example, a photoresist layer is formed on a silicon substrate, an example of a smooth substrate with homogeneous thickness, through a technique of spin coating, etc., and subjected to exposure and development via a predetermined mask pattern to form a template on the silicon substrate. The template includes concaves and convexes allowing transfer of the inner surface of the culture container 1, etc. Subsequently, an uncured curable resin composition or curable rubber composition is fed onto the template, and the composition is cured in the template, for example, through heating (involving pressurizing, in some cases) or UV irradiation. Then, the culture container 1, etc. cured is peeled off from the template. Variations on when to form concaves 4 and slits 5 are the same as for molding.

### 4. Other Embodiments

The concaves 4 open on the upper side are merely an example of rooms capable of individually receiving cells or embryos, and rooms each with a hole in the bottom or side wall are also acceptable. Although each slit 5 has a rectangular shape when viewed from the side wall of a concave 4, the shape may be another one such as an ellipse. The length of each slit 5 in the depth direction of the concaves 4 may be a length from the opening top of a concave 4 down to 50%, 40%, 30%, 20%, or 10% of the depth of the concave. The length of each slit 5 in the depth direction of the concaves 4 may be a length from the opening top of a concave 4 down to 60%, 70%, 80%, 90%, or 100% of the depth of the concave. The reserve tank 24 may be firmly fixed on the inside of the culture container 1, etc. The waste tank 25 may be a member attachable to and detachable from the culture container 1, etc.

### Examples

Now, Examples of the present invention will be described. However, the present invention is never limited to the following Examples.

### (1) Fabrication of Culture Container

Molds (a concave mold and a convex mold) were prepared for fabrication of a culture container having the shape of a Petri dish. The convex mold prepared included convexes as 5 rows×5 columns, or 25 convexes in total (diameter: 300 μm, height: 250 μm, convex-convex interval: 100 μm), sheets connecting concaves (width: 20 μm, height: 125 μm), and a protruding region (height: 1.5 mm, protruding area: 18 mm×10 mm). Into the mold, an uncured resin material (polystyrene) was fed with heating and pressurizing to cure, and the mold was then opened to afford a culture container having the shape of a Petri dish (diameter: 36 mm, height: 11 mm) and including concaves as 5 rows×5 columns, or 25 concaves in total (opening diameter: 300 μm, depth: 250 μm, concave-concave interval: 100 μm), slits (width: 20 μm, depth: 125 μm), and a waste tank (depth: 1.5 mm, opening: 18 mm×10 mm) in the inner surface. This culture container is referred to as “Vivo dish+50% slit”. In addition to the “Vivo dish+50% slit”, a culture container including 25 concaves independently formed without any slit (referred to as “Vivo dish+no slit”), and a culture container with the depth of each slit set to that of each concave, 250 μm (referred to as “Vivo dish+100% slit”) were each fabricated by using the above molding procedure except that the shape of the convex mold was changed.

### (2) Treatment Before Experiments

The culture containers fabricated in the above process and members including a reserve tank, which is described later, were sterilized with an electron beam or γ-ray in advance. Before introduction of fertilized eggs, each culture container was washed with KSOM (Lawitts J A, Biggers J D. Culture of preimplantation embryos. Methods Enzymol. 1993; 225: 153-64.). Thereafter, each culture container was placed in an incubator at 37° C. with an oxygen concentration of 5% and a carbon dioxide concentration of 5% until gas equilibrium was achieved.

Mouse 2-cell stage fertilized eggs were used for culture. A reserve tank (diameter: 10 mm, height: 15 mm) was disposed in a region out of the region including the dense population of concaves and the waste tank in each culture container, and culture solution was put therein. The culture solution put in the reserve tank was a solution prepared by adding 5% by mass human albumin to KSOM (Lawitts J A, Biggers J D. Culture of preimplantation embryos. Methods Enzymol. 1993; 225: 153-64.). Culture was performed in a CO2 incubator (5%02, 5% CO2, and 90% N2, 37° C., saturated humidity).

### (3) Experiments

### Experiment I: Effect of the Form of Culture Container Under Circulation of Culture Solution

Experiment Example (1)—The concaves of the culture container “Vivo dish+50% slit” were provided with the culture solution, and then the fertilized eggs were put in the concaves. Thereafter, the culture solution was fed from the reserve tank toward the concaves and slits of the culture container at a feeding rate of 10 nL/min to conduct circulation feeding leading to the waste tank. The development of each fertilized egg into a blastocyst was observed and recorded every 24 hours from 24 hours to 96 hours after the initiation of culture. The ratio of the number of fertilized eggs which developed into blastocysts to the number of the fertilized eggs at the initiation of culture (mouse 2-cell stage fertilized eggs) was determined as the development rate (%). The total cell count for cells constituting each of the resulting mouse blastocysts and the cell count for an inner cell mass in each blastocyst (ICM cell count) were acquired by using double fluorescence staining. The number of hatched blastocysts (blastocysts in a state of being hatched from the zona pellucida (a capsule covering a fertilized egg)) was counted similarly. Further, the pregnancy rate (%) in recipient females and offspring productivity (%) were determined.

Experiment Example (2)—Culture was performed in the same manner as Experiment Example (1) except that the culture container “Vivo dish+100% slit” was used instead of “Vivo dish+50% slit”.

Experiment Example (3)—Culture was performed in the same manner as Experiment Example (1) except that the culture container “Vivo dish+no slit” was used instead of “Vivo dish+50% slit”.

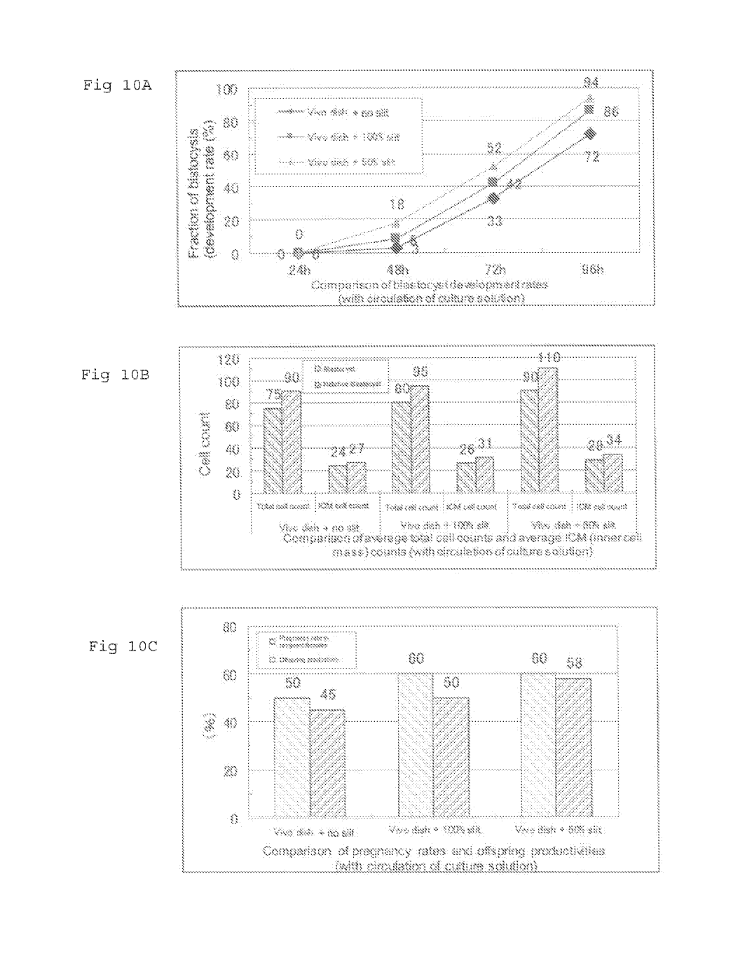

FIGS. 10A-10C show results of Experiment Examples (1) to (3), where the graph in FIG. 10A shows development rates (%) with different culture containers, the graph in FIG. 10B shows cell counts for different cells with different culture containers, and the graph in FIG. 10C shows pregnancy rates (%) in recipient females and offspring productivities (%) with different culture containers. In the graph in FIG. 10B, bars in the left side show results for blastocysts, and bars in the right side show results for hatched blastocysts. In the graph in FIG. 10C, bars in the left side show pregnancy rates (%) in recipient females, and bars in the right side show offspring productivities (%).

The culture containers with slits were superior to the culture container without any slit in any of development rates, cell counts, pregnancy rates (%) in recipient females, and offspring productivities (%). Differences depending on the slit depth were found as well, and the culture container with slits deep to the half of the depth of concaves was found to be superior to the culture container with slits deep to the whole depth of concaves in development rates, cell counts, pregnancy rates (%) in recipient females, and offspring productivities (%).

Experiment II: Effect of the Form of Culture Container without Circulation of Culture Solution

Experiment Example (4)—The concaves of the culture container “Vivo dish+50% slit” were provided with the culture solution, and then the fertilized eggs were put in the concaves. The development rate (%), cell counts, pregnancy rate (%) in recipient females, and offspring productivity (%) were determined in the same manner as in Experiment I.

Experiment Example (5)—Culture was performed in the same manner as Experiment Example (4) except that the culture container “Vivo dish+100% slit” was used instead of “Vivo dish+50% slit”.

Experiment Example (6)—Culture was performed in the same manner as Experiment Example (4) except that the culture container “Vivo dish+no slit” was used instead of “Vivo dish+50% slit”.

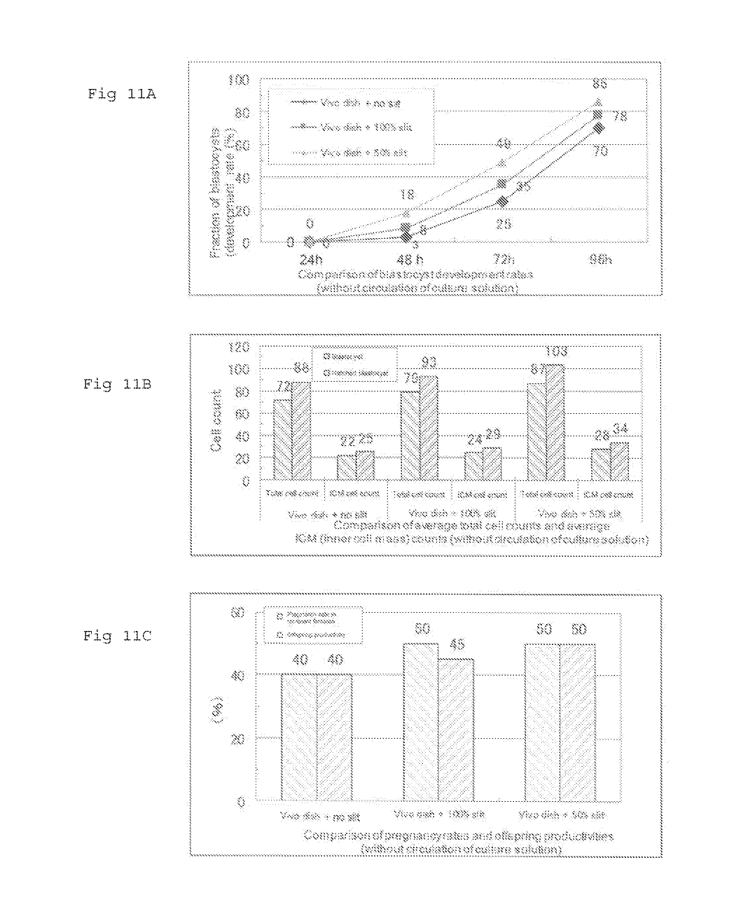

FIGS. 11A-11C show results of Experimental Examples (4) to (6), where the graph in FIG. 11A shows development rates (%) with different culture containers, the graph in FIG. 11B shows cell counts for different cells with different culture containers, and the graph in FIG. 11C shows pregnancy rates (%) in recipient females and offspring productivities (%) with different culture containers. In the graph in FIG. 11B, bars in the left side show results for blastocysts, and bars in the right side show results for hatched blastocysts. In the graph in FIG. 11C, bars in the left side show pregnancy rates (%) in recipient females, and bars in the right side show offspring productivities (%).

The culture containers with slits were superior to the culture container without any slit in any of development rates, cell counts, pregnancy rates (%) in recipient females, and offspring productivities (%). Differences depending on the slit depth were found as well, and the culture container with slits deep to the half of the depth of concaves was found to be superior to the culture container with slits deep to the whole depth of concaves in development rates, cell counts, pregnancy rates (%) in recipient females, and offspring productivities (%). Comparison with the results of Experiment Examples (1) to (3) shown above revealed that the development rates, cell counts, pregnancy rates (%) in recipient females, and offspring productivities (%) in Experiment Examples (4) to (6) were all lower than or comparable to those in Experiment Examples (1) to (3). These results demonstrate that it is preferred to culture with circulation of culture solution, it is more preferred to connect concaves via slits, and it is even more preferred that each slit be formed deep to the upper half of the depth of concaves, rather than deep to the whole depth of the depth of concaves.

### INDUSTRIAL APPLICABILITY

The present invention is applicable to culture of cells or embryos.

1. A culture container with an in vivo-like environment for culturing cells or embryos, comprising:
a plurality of rooms capable of individually receiving the cells or the embryos therein, wherein
two or more of the rooms are connected together via a space with a size allowing passage of culture solution but not allowing passage of the cells or the embryos.

2. The culture container with an in vivo-like environment of claim 1, wherein
the rooms are concaves open on one side, and
the culture container comprises slits each leading from an opening in a side wall of one of the concaves, the opening ranging from an upper position in the side wall to any position below, to the opening of a neighboring concave.

3. The culture container with an in vivo-like environment of claim 2, wherein the slits each have a length of 50% or less of a height of the concaves.

4. The culture container with an in vivo-like environment of claim 1, wherein a volume of each of the rooms is in a range of 10 to 50 nL.

5. The culture container with an in vivo-like environment of claim 1, further comprising:
a reserve tank that feeds culture solution into the culture container; and
a waste tank that stores culture solution discharged through the rooms.

6. A culture dish comprising the culture container with an in vivo-like environment of claim 1, comprising:
the culture container;
a reserve tank that feeds culture solution into the culture container; and
a waste tank that stores culture solution discharged from the culture container, wherein
the reserve tank, the culture container, and the waste tank are disposed at different vertical positions such that the culture solution in an inner space of the reserve tank moves from the reserve tank to the waste tank through the culture container.

7. The culture dish with an in vivo-like environment of claim 6, comprising a membrane between the reserve tank and the culture container, the membrane being capable of feeding the culture solution in the reserve tank to the culture container with a resistance to a gravity of the culture solution.
